(19) United States
(12) Patent Application Publication
(10) Pub. No.: US 2013/0237912 A1
(43) Pub. Date: Sep. 12, 2013

(54) BIOPSY MARKER DELIVERY DEVICE

(71) Applicant: DEVICOR MEDICAL PRODUCTS, INC., Cincinnati, OH (US)

(72) Inventor: Trevor W.V. Speeg, Williamsburg, OH (US)

(73) Assignee: Devicor Medical Products, Inc., Cincinnati, OH (US)

(21) Appl. No.: 13/873,533

(22) Filed: Apr. 30, 2013

Related U.S. Application Data
(63) Continuation of application No. 12/565,968, filed on Sep. 24, 2009, now abandoned.

(51) Int. Cl. A61M 5/42

(52) U.S. Cl. CPC A61M 5/427
USPC 604/116

(57) ABSTRACT

A biopsy marker delivery device includes a cannula, a grip supported adjacent the proximal end of the cannula, and at least one marker disposed within the internal lumen of the cannula. The cannula includes a distal tip closing the distal end of the cannula and a side marker exit port disposed proximally of the distal end of the cannula. The side marker exit port includes a first portion and a second portion extending proximally from the first portion. The first portion has a larger width than the second portion.

### PRIORITY

This application is a continuation of U.S. patent application Ser. No. 12/565,968, entitled “Biopsy Marker Delivery Device With Positioning Component,” filed Sep. 24, 2009, the disclosure of which is incorporated by reference herein.

### CROSS REFERENCE TO RELATED APPLICATIONS

This application cross references and incorporates by reference the following commonly assigned applications: U.S. patent application Ser. No. 12/196,301 filed Aug. 22, 2008; U.S. patent application Ser. No. 12/563,360 filed Sep. 21, 2009; U.S. patent application Ser. No. 12/365,390 filed Feb. 4, 2009; and U.S. patent application Ser. No. 12/406,135 filed Mar. 18, 2009.

### BACKGROUND

Biopsy samples have been obtained in a variety of ways in various medical procedures using a variety of devices. An exemplary biopsy device is the MAMMOTOME.®. brand device from Ethicon Endo-Surgery, Inc. of Cincinnati, Ohio. Biopsy devices may be used under stereotactic guidance, ultrasound guidance, MRI guidance, or otherwise.

Further exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2007/0118048, entitled “Remote Thumbwheel for a Surgical Biopsy Device,” published May 24, 2007; U.S. Provisional Patent Application Ser. No. 60/869,736, entitled “Biopsy System,” filed Dec. 13, 2006; U.S. Provisional Patent Application Ser. No. 60/874,792, entitled “Biopsy Sample Storage,” filed Dec. 13, 2006; and U.S. Non-Provisional patent application Ser. No. 11/942,785, entitled “Revolving Tissue Sample Holder for Biopsy Device,” filed Nov. 21, 2007. The disclosure of each of the above-cited U.S. patents, U.S. patent application Publications, U.S. Provisional patent applications, and U.S. Non-Provisional patent application is incorporated by reference herein.

In some settings, it may be desirable to mark the location of a biopsy site for future reference. For instance, one or more markers may be deposited at a biopsy site before, during, or after a tissue sample is taken from the biopsy site. Exemplary marker deployment tools include the MAMMOMARK.®., MICROMARK.®., and CORMARK.®. brand devices from Ethicon Endo-Surgery, Inc. of Cincinnati, Ohio. Further exemplary devices and methods for marking a biopsy site are disclosed in U.S. Pub. No. 2005/0228311, entitled “Marker Device and Method of Deploying a Cavity Marker Using a Surgical Biopsy Device,” published Oct. 13, 2005; U.S. Pat. No. 6,996,433, entitled “Imageable Biopsy Site Marker,” issued Feb. 7, 2006; U.S. Pat. No. 6,993,375, entitled “Tissue Site Markers for In Vivo Imaging,” issued Jan. 31, 2006; U.S. Pat. No. 7,047,063, entitled “Tissue Site Markers for In Vivo Imaging,” issued May 16, 2006; U.S. Pat. No. 7,229,417, entitled “Methods for Marking a Biopsy Site,” issued Jun. 12, 2007; U.S. Pat. No. 7,044,957, entitled “Devices for Defining and Marking Tissue,” issued May 16, 2006; U.S. Pat. No. 6,228,055, entitled “Devices for Marking and Defining Particular Locations in Body Tissue,” issued May 8, 2001; and U.S. Pat. No. 6,371,904, entitled “Subcutaneous Cavity Marking Device and Method,” issued Apr. 16, 2002. The disclosure of each of the above-cited U.S. patents and U.S. patent application Publications is incorporated by reference herein.

It may be desirable to deploy markers from a cannula type deployer into the biopsy site, such as a flexible tubular deployer. The marker should not unintentionally fall out of the deployer, and the force to deploy the marker should not be excessive. Further, the tubular deployer should not advance further within the biopsy device than intended.

### SUMMARY

In one non-limiting aspect, the present invention provides a biopsy marker deployer comprising a tube carrying at least one biopsy marker, and inner pushing member such as push rod. The push rod is disposed within the outer tube and is advanceable within the tube to urge the marker out of the deployer.

Applicant has recognized the desirability of avoiding “over advancing” the deployer, and particularly the deployer tip, into the associated biopsy device when deploying markers through a biopsy device.

Applicant has further recognized the desirability of providing a feature on the outside of the deployer tube (or shaft), the feature disposed a predetermined distance from the distal tip of the deployer.

According to one aspect of the present invention, a method is provided. The method may include the steps of obtaining a hollow tube having a sidewall, a proximal end, a distal end, and an internal lumen; forming an endpiece in place in the distal end of the hollow tube to close the distal end of the tube; forming a component extending around an outside surface of the hollow tube, wherein the step of forming the component comprises positioning the component along the tube at a predetermined distance from the endpiece between proximal end of the hollow tube and the distal end of the hollow tube; and disposing at least one biopsy marker in the internal lumen.

The method may further comprise forming an opening in the side wall of the tube between the distal end of the hollow tube and the component, the opening providing an exit through which a marker may be deployed.

In another aspect of the present invention, a biopsy marker delivery device is provided for deploying biopsy markers. The device may include: a cannula having an internal lumen extending from a proximal end of the cannula to the distal end of the cannula, and a marker exit formed in a sidewall of the cannula proximal to the distal end of the cannula; a distal tip closing the distal end of the cannula; a component disposed on the outer surface of the cannula sidewall, the component disposed at a position along the length of the cannula between the proximal end of the cannula and the marker exit formed in the sidewall of the cannula, and the component being sized and shaped to limit the depth to which the cannula can be inserted within a biopsy instrument; and at least one marker disposed within the internal lumen of the cannula.

The component may be annular in shape and may be disposed at a position along the length of the cannula at a predetermined distance from the distal tip. The component may be overmolded on the cannula, and fixed with respect to the cannula. The component may further include an proximally extending tab circumferentially aligned with the marker exit port.

The component may be positioned a predetermined distance between a grip (disposed at or near the proximal end of the cannula) and at least one of the marker exit and the tip. The component may have an annular body having an axial length of less than about 0.5 inch and an axially extending tab, the tab extending proximally from the annular body, and the tab having a length at least about twice the axial length of the annular body.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of a marker delivery device of the type illustrated in U.S. patent application Ser. No. 12/196,301 filed Aug. 22, 2008.

FIG. 2 depicts a cross-sectional view of a distal portion of a marker delivery device of the type illustrated in U.S. patent application Ser. No. 12/196,301 filed Aug. 22, 2008.

FIG. 3 depicts a marker being deployed from a deployer and through a lateral tissue receiving port in a biopsy needle to mark a biopsy site, such as illustrated in U.S. patent application Ser. No. 12/196,301 filed Aug. 22, 2008.

FIG. 4 depicts a portion of a marker deployment tube is shown cutaway to illustrate the inner diameter of the tube is generally smooth, and to reveal a member, such as a push rod, the push rod having a surface feature effective for reducing the contact surface area between a portion of the push rod disposed within the deployment tube and the inner surface of the deployment tube, and FIG. 4 showing a push rod having a surface finish and surface roughness different than those of the inner diameter of the tube, the push rod shown having a plurality of generally longitudinally extending ribs having peaks elevated above relatively lower elevation depressions.

FIG. 5. Depicts a cross-section of the pushing member and illustrating the peaks of the elevated portions of the longitudinally extending ribs in relation to the diameter of the pushing member and in relation to the recessed portions of the outer surface of the push rod.

FIG. 6 depicts a cross-section of the deployer with the pushing member shown disposed within the deployer tube, and illustrating the circumferential peak to peak spacing of adjacent longitudinally extending ribs can be greater than the radial height of the ribs.

FIG. 7 illustrates a pushrod having a relatively rigid proximal portion 18A (such as stiffened by a metal sleeve) and a relatively flexible distal portion 18B comprising a plurality of longitudinally extending ribs.

FIG. 8 illustrates a plurality of ring like ribs providing spaced apart raised surfaces disposed on the outer surface of a push rod, adjacent ring like ribs spaced longitudinally from one another along a portion of the push rod disposed within the deployment tube.

FIG. 9 illustrates an embodiment having a surface feature on the inside surface of the cannula lumen.

FIG. 10 is a schematic illustration showing a marker delivery device according to one embodiment of the present invention, and showing a component, such as a positioning component having a generally annular body and a proximally extending member, the component positioned at predetermined distance from the tip of the delivery device, and the component positioned at a predetermined distance with respect to a side marker exit.

FIG. 11 is an enlarged schematic illustration of a distal portion of the device of FIG. 10, FIG. 11 illustrating a distal tip and a side marker exit port disposed proximally of the distal tip, the exit port having a proximal portion and a relatively larger distal portion.

FIG. 12 is a schematic illustration of the component shown in FIG. 10 positioned with respect to an opening in a biopsy device, FIG. 12 illustrating an alignment feature, such as a distally extending male alignment feature extending from the annular body of the positioning component for engagement with a female alignment feature, such as an alignment groove, formed in portion of a biopsy device.

FIG. 13 is a schematic illustration of a biopsy marker delivery device according to an embodiment of the present invention, FIG. 13 providing a top view of the biopsy marker delivery device and illustrating alignment of a positioning component with the biopsy marker exit.

FIG. 14 is a schematic illustration of the biopsy marker device of FIG. 13, FIG. 13 providing a side view of the biopsy device of FIG. 13 and illustrating predetermined spacing of a positioning component with respect to the distal tip of the device, such as where the positioning component and the distal tip are molded in place with respect to the cannula of the delivery device in the same molding operation.

FIG. 15 is a schematic illustration of a biopsy marker delivery device according to an embodiment of the present invention positioned for insertion into a proximal opening in a proximal end of a biopsy device.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

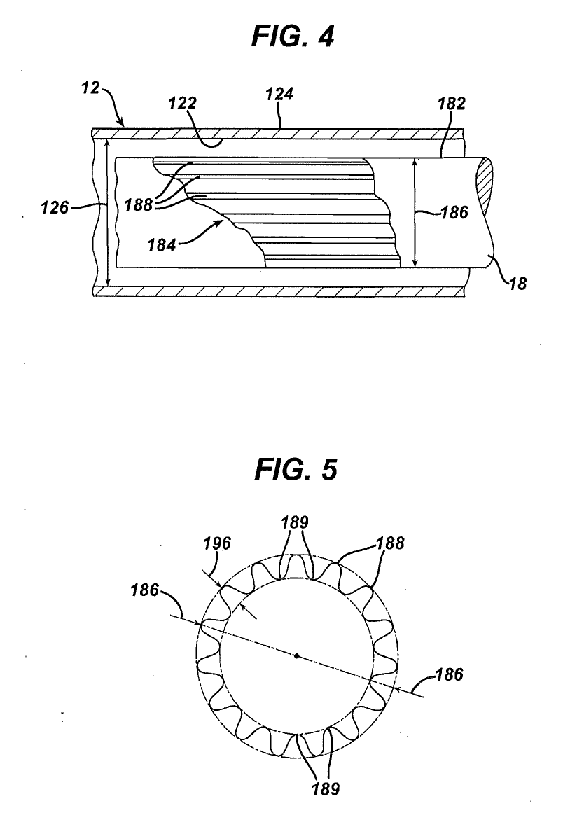

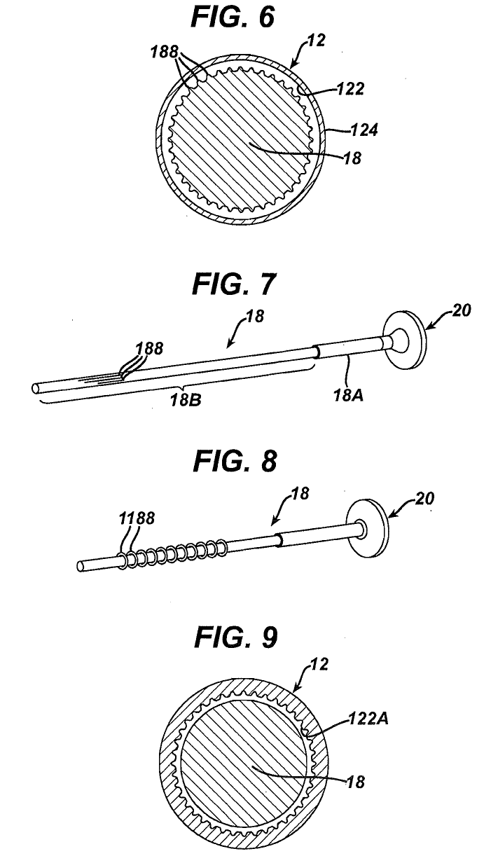

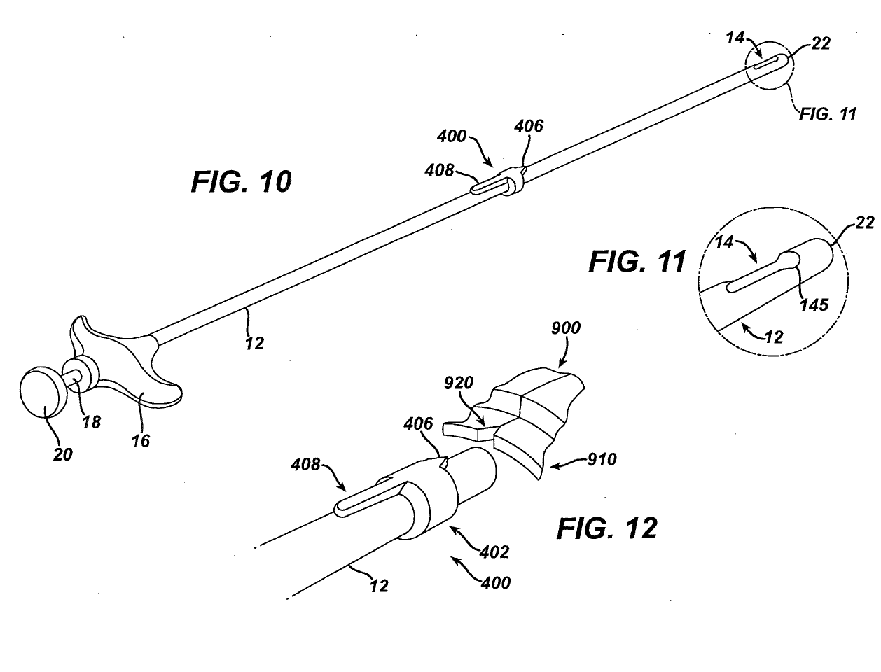

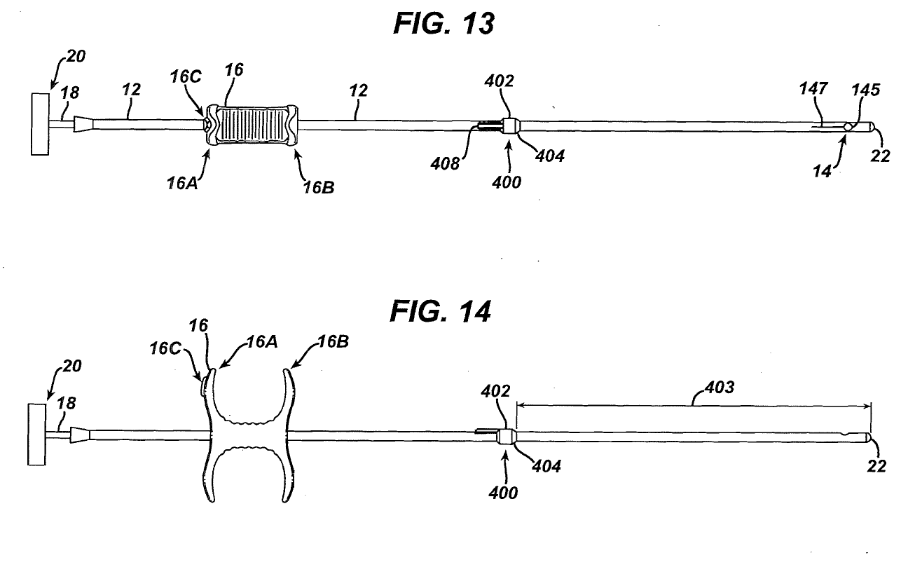

FIGS. 13 illustrate a marker delivery device (10) of the type illustrated in copending, commonly assigned U.S. patent application Ser. No. 12/196,301 filed Aug. 22, 2008. Marker delivery device (10) may include a tubular elongate outer cannula (12) having a marker exit, such as side opening (14) formed near to, but spaced proximally from, distal end of cannula (12). FIGS. 4-9 illustrate various features of a biopsy marker delivery device of the type disclosed in copending, commonly assigned U.S. patent application Ser. No. 12/563,360 filed Sep. 21, 2009. FIGS. 10-15 illustrate one or more biopsy marker delivery devices according to the present invention.

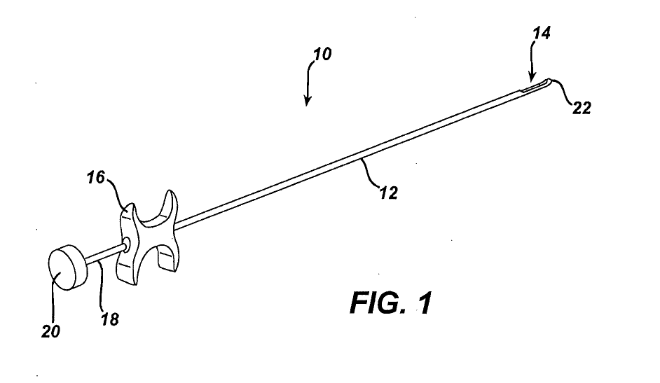

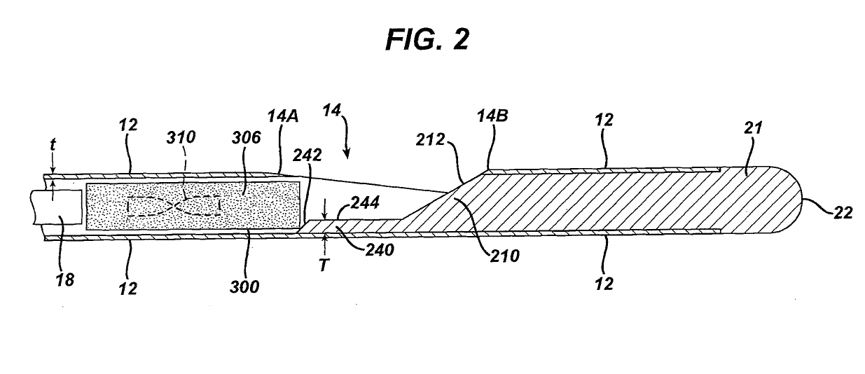

Referring to FIGS. 1-3, a grip (16) can be provided at the proximal end of cannula (12). A pushing member in the form of a push rod (18) can be provided, with push rod (18) extending coaxially in cannula (12) such that push rod (18) is configured to translate within cannula (12) to displace one or more markers through side opening (14) (see FIG. 2). Rod (18) can have a proximal portion (proximal portion 18A in FIG. 7) have sufficient rigidity in compression to push a marker from the internal lumen of cannula (12) out through opening (14), and include a more distal portion (for example portion 18B in FIG. 7) that is relatively flexible in bending so that cannula (12) can be inserted along a curved path to deploy a marker element at a biopsy site.

A plunger (20) can be provided at the proximal end of rod (18) for forcing rod (18) distally in cannula (12) to deploy a marker out of cannula (12). A user may grasp grip (16) with two fingers, and may push on plunger (20) using the thumb on the same hand, so that marker delivery device (10) can be operated by a user's single hand. A spring (not shown) or other feature may be provided about rod (18) to bias rod (18) proximally relative to grip (16) and cannula (12).

FIG. 2 depicts a crosssectional view of a distal portion of marker delivery device (10). FIG. 2 shows a biopsy marker (300) disposed in internal lumen (15) of cannula (12). The marker (300) can comprise a biodegradable or otherwise resorbable body (306), such as a generally cylindrically shaped body of collagen, and a metallic, generally radiopaque marker element (310) (shown in phantom) disposed within or otherwise carried by body (306).

The cannula (12) can be formed of any suitable metallic or nonmetallic material.

In one embodiment, cannula (12) is formed of a thin walled hollow tube formed of a suitable medical grade plastic or polymer. One suitable material is a thermoplastic elastomer, such as Polyether block amide (PEBA), such as is known under the tradename PEBAX. The cannula (12) can be formed of PEBAX, and can be substantially transparent to visible light and X-ray.

The side opening (14) can be formed by cutting away a portion of the wall of cannula (12). The side opening (14) communicates with an internal lumen (15) of the cannula. The side opening (14) can extend axially (in a direction parallel to the axis of lumen (15)) from a proximal opening end (14A) to a distal opening end (14B), as illustrated in FIG. 2.

The distal tip (22) extending from the distal end of cannula (12) can be rounded as shown in FIG. 2. Referring to FIG. 2, a marker delivery device can have the distal end of cannula (12) closed by a unitary endpiece (21) formed in place in the distal end of cannula (12), with a part of endpiece (21) extending into internal lumen (15) of the cannula. The distal endpiece (21) can be a molded or cast component, and can provide an integrally formed combination of tip (22), a ramp (210) having a ramp surface (212), and a marker engaging element (240). The ramp surface (212) aids in directing marker (300) from internal lumen (15) through side opening (14). The marker engaging element (240) may be employed to retain marker (300) in internal lumen (15) until the user intends to deploy the marker.

The marker engaging element (240) may be disposed within internal lumen (15), and at least a portion of the marker engaging element is disposed distally of the proximal end (14A) of side opening (14). The marker engaging element (240) can extend along a portion of the floor of cannula (15) under opening (14), and marker engaging element (240) can be positioned to reinforce the portion of the cannula in which opening (14) is formed. For instance, by positioning marker engaging element (240) underneath opening (14), as shown in FIG. 2, element (240) can help to stiffen cannula (12) in the region where wall of cannula (12) is cut to form opening (14). In FIG. 2, marker engaging element (240) extends from the proximal most portion of ramp surface (212), and does not extend proximally of side opening (14), though in other embodiments, a portion of element (240) could extend proximally of opening (14).

In the embodiment shown in FIG. 2, marker engaging element (240) is in the form of a step having a generally uniform thickness T along the element's axial length, except that the element has a tapered proximal end (242). The tapered proximal end (242) can form an included angle with the longitudinal axis of lumen (15) (included angle with a horizontal line in FIG. 2) of about 45 degrees, while ramp surface (212) can form an included angle with the longitudinal axis of about 30 degrees.

The thickness T can be greater than the wall thickness t of cannula (12), and in one embodiment T is at least about twice the thickness t. In one embodiment, the thickness T can be between about 0.018 inch to about 0.040 inch, and the wall thickness t can be between about 0.005 inch to about 0.008 inch. The internal diameter of lumen (15) can be about 0.120 inch.

In FIG. 2, upwardly facing surface (244) (surface facing opening (14)) marker engaging element (240) extends distally to contact ramp surface (212), so that there is not a space or gap between surface (244) and ramp surface (212). Such an arrangement is advantageous to reduce the possibility that marker (300), upon moving past the marker engaging element, will become lodged between the marker engagement element and the ramp.

If desired, marker engaging element (240), ramp (210), and/or tip (22) can be formed of, or include, a material that is relatively more radiopaque than the wall of cannula (12). For instance, where element (240), ramp (210), and tip (22) are formed as an integral endpiece (21), endpiece (21) can include a radiopaque additive, such as barium sulfate. For instance, endpiece (21) can be a component molded of PEBAX, with about 20 percent by weight barium sulfate added to the molten PEBAX mold composition.

The relatively more radiopaque marker engaging element (240), ramp (210), and tip (22) can be useful in distinguishing the position of those components using radiographic imaging. Also, where the ramp and/or step of engaging element are positioned in association with opening (14), the addition of a radiopaque material can help identify the position of the opening, and the position of marker (300) relative to the opening before, during, or after deployment of the marker.

Only one marker is shown disposed in lumen (15) in the figures. However, it will be understood that multiple markers can be disposed in marker delivery device (10), such as in an end to end configuration. The markers can have the same size and shape, or alternatively have different sizes and/or shapes.

The cannula (15) can be generally transparent to visible light and x-ray, and endpiece (21) can be generally opaque to visible light and x-ray. If desired, endpiece (21) can be colored with a dye or other suitable colorant in the liquid mold composition. For example, it may be desirable to have different size markers (e.g. length and/or diameter) for different biopsy procedures. For instance, it may be desirable to provide a larger marker if a relatively large biopsy sample is taken, and a smaller marker if a relatively small biopsy sample is taken. The endpiece (21) can be colored using one of multiple colors to indicate the size of the marker disposed in the cannula. For instance, if three marker sizes are provided, endpiece (21) can be colored one of three colors to identify which of the marker sizes are disposed in the cannula of a particular marker device. The endpiece (21) can also be colored to indicate a particular size (diameter or length) biopsy needle with which the marker delivery device is to be used. Additionally, multiple marker delivery devices could be packaged in kit form, with the kit including marker delivery devices having different size markers and correspondingly colored end pieces.

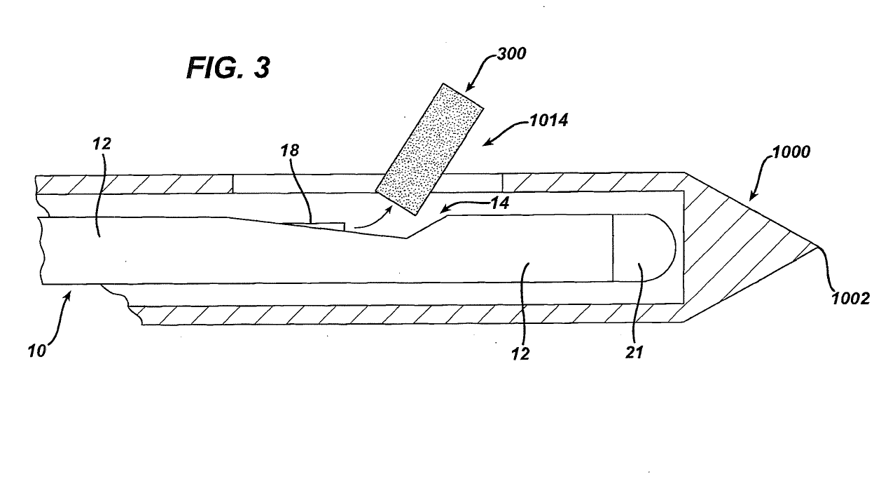

Referring to FIG. 3, marker delivery device (10) may be used to deploy a marker to mark a particular location within a patient. In FIG. 3, a cannular biopsy needle (1000) is shown. The needle (1000) is shown having a closed distal end with piercing tip (1002), and a lateral tissue receiving aperture (1014). Marker deployer (10) may be introduced to a biopsy site through biopsy needle (1000), which can be the same needle used to collect a tissue sample from the biopsy site. The biopsy needle (1000) can be of the type used with single insertion, multiple sample vacuum assisted biopsy devices. Several such biopsy devices are disclosed in the various patents and patent applications that have been referred to and incorporated by reference herein, though other biopsy devices may be used.

FIG. 3 shows the distal end of a marker deployer (10) disposed within needle (1000). The needle (1000) can be positioned in tissue, and a biopsy sample can be obtained through opening (1014), thereby providing a biopsy cavity adjacent opening (1014). Then, after the tissue sample has been obtained and transferred proximally through the needle, and without removing needle (1000) from the patient's tissue, deployer (10) can be inserted into a proximal opening in needle (1000). In FIG. 3, needle (1000) and deployer (10) are positioned such that opening (14) of cannula (12) and opening (1014) of needle (1000) are substantially aligned axially and circumferentially. Then, with the deployer and needle so positioned at the biopsy site, push rod (18) can be advanced to deploy the marker up ramp surface (212), through opening (14), and then through opening (1014), into the biopsy cavity.

In some instances, it may be necessary to bend or otherwise flex marker deployer cannula (12) and push rod (18) when inserting the deployer into the biopsy device. By reducing the effective contact surface area between the outer surface of push rod (18) and the inner surface of cannula (12), Applicants believe the tendency of push rod (18) to “lock” within cannula (12) can be reduced and/or eliminated.

Referring now to FIGS. 4-9, FIG. 4 illustrates a portion of cannula (12) and push rod (18), with part of cannula (12) cut away to show push rod (18) disposed within cannula (12). The cannula (12) can be formed from a thin wall, flexible nonmetallic tube having a generally smooth outer surface (124), a generally smooth inner surface (122), and having an inner diameter designated (126) in FIG. 4. A generally flexible, elongate pushing member, such as a portion of push rod (18), is disposed at least partially within the internal lumen of the hollow cannula (12). The push rod (18) has an outer diameter designated (186) in FIG. 4.

In FIG. 4, push rod (18) is illustrated having an outer surface (182) that has a surface feature designated generally as (184), which surface feature is effective for reducing the contact surface area between the outer surface of push rod (18) and the inner surface of the lumen extending through cannula (12) when cannula (12) and rod (18) are bent or otherwise flexed. In one embodiment, surface feature (184) is configured to be effective in providing at least about a 50 percent reduction (still more particularly at least about 75% reduction) in the contact surface area that would otherwise occur for a push rod (18) and cannula (12) both having generally smooth, untextured surfaces and the same nominal outer diameter and inner diameter.

In the embodiment shown in FIG. 4, surface feature (184) is shown comprising a plurality of longitudinally extending elevated portions in the form of ribs (188). The ribs (188) extend along at least a portion of push rod (18) disposed within cannula (12).

For marker deployers (10) useful in connection with breast biopsy devices having a breast biopsy needle, and useful for deploying breast biopsy markers from breast biopsy devices, inner diameter (126) of lumen of cannula (12) may be (but is not limited to) at least about 0.08 inch, and outer diameter (186) of push rod (18) may be (but is not limited to) between about 0.04 inch and about 0.09 inch.

In one embodiment, ribs (188) can have a radial height (196) measured with respect to adjacent recessed portions (designated as valleys (189)) of between about 0.0001 inch and about 0.01 inch. More particularly, ribs (188) can have a radial height of between about 0.0003 inch and about 0.004 inch, yet more particularly, radial height (196) can be between about 0.0005 inch and about 0.004 inch. In one non-limiting example, radial height (196) can be between about 0.001 inch and about 0.003 inch, such as about 0.002 inch plus or minus 0.001 inch. The radial height (196) can be less than one tenth of diameter (186) of the push rod, and more particularly less than about one twentieth of diameter (186). The radial height (196) can be less than one half (less than 50 percent of), and more particularly less than about one quarter of the difference between outer diameter (186) and inner diameter (126) of the lumen of cannula (12).

The number and size of longitudinal surface features may be selected to be effective in reducing the effective contact surface area between push rod and the inner surface of the cannula, without interfering with sliding of the push rod within the lumen of the cannula. For instance, but without being limited by theory, in one embodiment push rod (18) may have at least about 20 ribs spaced around it's circumference, and less than about 100 ribs. The ribs can be formed by extruding, molding, or other suitable methods. The circumferential spacing between adjacent ribs can be greater than radial height (196) of the adjacent ribs.

In one non limiting example, a biopsy marker deployer (10) of the present invention suitable for use through an 11 gauge breast biopsy needle can have a push rod diameter (186) of about 0.060 inch, a cannula inner diameter (126) of about 0.084 inch, and about 40-50 splines spaced around the circumference of the push rod, the splines being generally uniformly spaced apart and having a radial height of about 0.002 inch. Without being limited by theory, it is believed that such a configuration can be effective in reducing the effective contact area between cannula (12) and rod (18) to about 0.246 square inch from about 1.158 square inch.

In another non limiting example, a biopsy marker deployer (10) of the present invention suitable for use in an 8 gauge breast biopsy needle can have a push rod diameter (186) of about 0.082 inch, a cannula inner diameter (126) of about 0.120, and about 50-70 splines spaced around the circumference of the push rod, the splines being generally uniformly spaced apart and having a radial height of about 0.002 inch. Without being limited by theory, it is believed that such a configuration can be effective in reducing the effective contact area between cannula (12) and rod (18) to about 0.388 square inch from about 1.665 square inch.

FIG. 7 illustrates a push rod (18) having a relatively stiff proximal section (18A), and a flexible portion (18B) comprising a plurality of ribs (188) as described above. The relatively stiff proximal portion (18A) can comprise a metallic sleeve or other stiffening member disposed at the proximal end of the rod to prevent the proximal end of the push rod from bending or kinking when plunger (20) is pressed to deploy a marker. The flexible portion (18B) may comprise ribs (188) or other surface features along some or substantially all the length of flexible portion (18B) such as to be effective in preventing locking of push rod (18) within cannula (12) when the rod and cannula are bent or otherwise disposed along a curved path.

FIG. 8 illustrates surface features (1188) disposed at spaced apart locations along the length of push rod (18). The surface features (1188) may be in the form of longitudinally spaced apart raised rings extending circumferentially around the diameter of push rod (18). The rings may be circumferentially continuous or formed of discrete segments. In yet another alternative embodiment, the outer surface of push rod (18) may comprise surface features in the form of bumps or protrusions, such as bumps or protrusions having the radial height characteristics set forth above. The bumps or protrusions may be randomly positioned on the surface of rod (18), or may be arranged in a predetermined pattern.

FIG. 9 illustrates cannula (12) having an inner surface (122A) having a surface feature effective for reducing binding/locking of rod (18) within cannula (12).

FIGS. 10-15 illustrate a biopsy marker delivery device according to the present invention. In FIG. 10, a biopsy marker delivery device is illustrated including a tubular elongate outer cannula (12), a marker exit, such as a side marker exit (14) formed in a sidewall of cannula (12), a grip (16), a push rod (18), and a plunger (20). In addition, the biopsy marker delivery device in FIG. 10 includes a positioning component (400).

The positioning component (400) is shown disposed on the outer surface of the cannula sidewall, and is positioned along the length of the cannula between the proximal end of the cannula and marker exit (14). In FIG. 10, component (400) is shown positioned around the outer surface of cannula (12) at a longitudinal position intermediate marker exit (14) and grip (16). The component (400) is sized and shaped to limit the depth to which cannula (12) may be inserted into a biopsy instrument, such as a single insertion, multiple sample vacuum assisted biopsy device.

In one embodiment, component (400) is formed on cannula (12), such as by molding, to be stationary with respect to cannula, and such that component is positioned a predetermined fixed distance from distal tip (22) of endpiece (21) and marker exit (14).

FIG. 11 provides a schematic, enlarged view of a distal portion of the biopsy marker delivery device in FIG. 10. As shown in FIG. 11, the marker exit may optimally include a relatively smaller proximal portion, and a relatively enlarged distal portion (145).

FIG. 12 provides a schematic illustration of a portion of the biopsy marker delivery device (including component (400)) positioned with respect to a proximal end (910) of a biopsy device (900). Biopsy device (900) may be a biopsy device of the type including a needle (1000) (FIG. 3). For instance, biopsy device (900) may be a single insertion, multiple sample vacuum assisted biopsy device. Cannula (12) may be sized to fit through a proximal opening in proximal end (910) of biopsy device (900), and component (400) may be sized and shaped to ensure that cannula (12) is not over inserted into device (900). For instance, component (400) may be sized and shaped to ensure that end piece (21) of the marker delivery device does not extend out of needle (1000) of biopsy device (900).

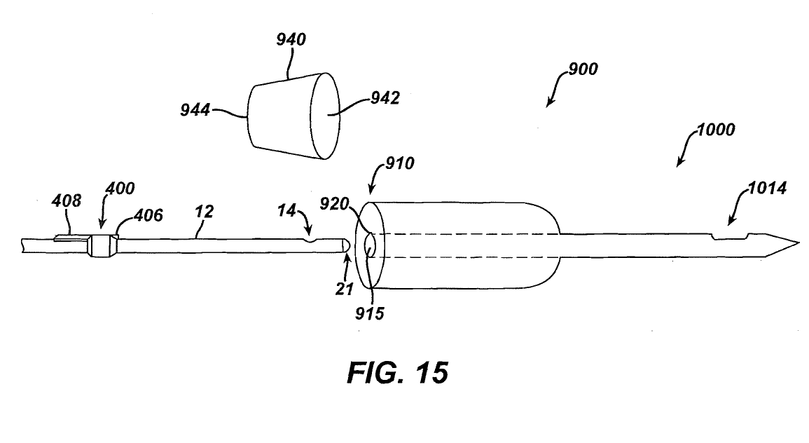

FIG. 15 illustrates schematically how the marker delivery device having a component (400) may be inserted through a proximal opening (915) at a proximal portion (910) of a biopsy device (900) having needle (1000). The marker delivery device may be inserted through opening (915) and advanced within biopsy device (900), with component (400) permitting exit (14) to be registered with needle opening (1014) (as seen in FIG. 3), while component (400) prevents over insertion of cannula (12) in biopsy device (900). In FIG. 15, a proximal cover (940) is shown removed from proximal portion (910) to expose opening (915). Opening (915) may communicate directly or indirectly with the lumen of needle (1000). Cover (940) may be in the form of a cup or tissue sample holder having an open distal end (942) and a closed proximal end (944), cover (940) being releasable with respect to proximal portion (910), and cover (940) covering opening (915) during biopsy sampling.

Referring to FIGS. 10-15, component (400) may include a generally annular shaped body (402). The component (400) may also include a proximally extending portion, such as a proximally extending tab (408) shown in FIGS. 10 and 12. The proximally extending tab (408) is shown to be aligned circumferentially with respect to (generally same o'clock position as) marker exit (14). The annular body (402) can include a distally facing tapered surface (404), and an alignment feature, such as a distally extending male alignment feature (406) that engages with a female alignment feature (920) of biopsy device (900). Engagement of feature (406) with feature (920) helps maintain alignment of marker exit (14) with needle side opening (1014).

As shown in FIG. 14, positioning component (400) is spaced a predetermined axial (longitudinal) distance (403) from distal tip (22) of end piece (21). The distance (403) may be provided by overmolding component (400) on the outer surface of cannula (12).

In one embodiment, component (400) and end piece (21) may be formed together, such as by molding component (400) over cannula (12) and molding end piece (22) in the distal end of cannula (12) in the same mold and during the same mold operation. For instance, the hollow tube may be placed within a mold having a mold cavity corresponding to component (400) and a mold cavity corresponding to end piece (21).

The biopsy marker delivery device may be formed by obtaining a thin wall, flexible tube for use as cannula (12), forming end piece (21) in the distal end of the tube, such as by molding, to close the distal end of the tube; overmolding the positioning component (400) on the outer surface of the hollow tube a predetermined distance from the distal end of the tube; such that component (400) is positioned along the tube at a predetermined distance from distal tip (22) of end piece (21), and positioned between the proximal end of the hollow tube and the distal end of the hollow tube. The marker exit (14) may be formed by cutting or otherwise forming an opening in the sidewall of the tube. After end piece (21) and component (400) are molded on the tube, at least marker may be positioned with the lumen of the tube through the proximal end of the tube.

In another embodiment, component (400) may be formed separately, and then slid over cannula (12). In such an embodiment, component (400) may be fixed with respect to cannula (12), or alternatively, be sized to slide along the length of the cannula (12) so that component (400) may be positioned at one or more axial markings or indicia spaced along the length of the tube. For instance, cannula (12) may have a plurality of axial markings along its length, each marking associated with a different size/length of biopsy needle (1000). The user may then slide component (400) to the desired indicia corresponding to biopsy device (900)/needle (1000) into which cannula (12) is to be inserted, thereby ensuring that cannula (12) is inserted to the correct depth with respect to biopsy device (900)/needle (1000) being used.

The component (400) may be sized such annular body (402) has an axial length of less than about 1 inch, and more particularly less than about 0.5 inch. The proximally extending tab (408) can have a length at least about twice the axial length of the annular body (402). In one non-limiting embodiment, the axial length of body (402) can be about 0.2 to about 0.3 inch, the outer diameter of body (402) can be about 0.15 inch to about 0.16, the inner diameter of body (402) can be about 0.09 to about 0.11 inch, and the combined axial length of body (402) and tab (408) can be about 0.7 to about 0.8 inch.

Referring to FIGS. 13 and 14, the biopsy marker delivery device may have a marker exit (14) comprising a proximal portion in the form of a narrow opening or slit (147), and a distal portion in the form of a circular or oval opening (145). The proximal tab (408) may be aligned with exit (14), as well as with finger extensions (16A) and (16B) of grip (16). An orientation mark, such as an arrow shaped raised portion (16C) on finger extension (16A), may also be aligned with proximally extending tab (408).

If desired, grip (16) and/or positioning component (400) and/or end piece (21) may be formed of the same material (such as a non-metallic material) and/or have the same color, such as where the color is indicative of a particular gauge size needle into which the marker delivery device is meant to be inserted. In one embodiment, the component (400) and end piece (21) are molded (such as by being molded together in the same mold and/or during the same molding operation) using the same material and with the same color, depending on the needle gauge for which the biopsy marker delivery device is intended to be used. By way of example, the component/end piece could be molded with a green material for 8 gauge applications, a blue color for 11 gauge applications, and a red color for 14 gauge applications. Additionally, a kit may be provided having multiple marker delivery devices packaged together, at least some of the marker delivery devices being sized to be inserted in different gauge needles (1000).

In another embodiment, end piece (21) and/or component (400) may be attached to cannula (12), such as by an adhesive, by heat bonding, ultrasonic welding, or other suitable methods.

The present invention has been disclosed with respect to a biopsy marker deployer device. However, the various features and components disclosed in the figures may be employed in devices useful with radioisotope applications, as in PEM, BSGI, and other imaging methods that may employ a radioisotope or other radiation source in connection with imaging a biopsy procedure.

Embodiments of the devices disclosed herein are generally designed to be disposed of after a single use, but could be designed to be used multiple times. After forming the marker, and inserting the marker into the deployer, the biopsy device can be sterilized. The device can be placed in a package, such as plastic or TYVEK bag.

The packaged biopsy device may then be placed in a field of radiation such as gamma radiation, x-rays, or high-energy electrons to sterilize the device and packaging. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. Accordingly, the scope of the present invention should be considered in terms of the following claims and is understood not to be limited to the details of structure and operation shown and described in the specification and drawings.

I/we claim:

1. A biopsy marker delivery device comprising:
(a) a cannula comprising:
(i) a proximal end,
(ii) a distal end, wherein the distal end comprises a distal tip closing the distal end of the cannula,
(iii) an internal lumen extending from the proximal end to the distal end, and
(iv) a side marker exit port disposed proximally of the distal end of the cannula, wherein the side marker exit port is in fluid communication with the internal lumen, wherein the side marker exit portion comprises a first portion and a second portion extending proximally from the first portion, wherein the first portion defines a larger width than the second portion;
(b) a grip supported adjacent the proximal end of the cannula; and
(c) at least one marker disposed within the internal lumen of the cannula.

2. The biopsy marker delivery device of claim 1, wherein the first portion is generally circular.

3. The biopsy marker delivery device of claim 1, wherein the second portion defines a slit.

4. The biopsy marker delivery device of claim 1, wherein the first portion is non-circular.

5. The biopsy marker delivery device of claim 1, wherein the cannula comprises a ramp operatively associated with the side marker exit port.

6. The biopsy marker delivery device of claim 1, further comprising a component that is slidable along the cannula.

7. The biopsy marker delivery device of claim 1, wherein the distal tip comprises a non-metallic material.

8. The biopsy marker delivery device of claim 1, wherein the grip is operable to rotate the cannula.

9. The biopsy marker delivery device of claim 1, further comprising a push rod disposed within the cannula and having a closed distal end, wherein the push rod is operable to advance the at least one marker through the marker exit.

10. A biopsy marker delivery device for use with a biopsy instrument, wherein the biopsy marker delivery device comprises:
(a) a cannula comprising:
(i) a proximal end,
(ii) a distal end,
(iii) an internal lumen extending from the proximal end to the distal end, and
(iv) a side marker exit port disposed proximally of the distal end of the cannula, wherein the side marker exit port is in fluid communication with the internal lumen, wherein the side marker exit portion comprises a first portion and a second portion adjacent to the first portion, wherein the second portion defines a slit; and
(b) at least one marker disposed within the internal lumen of the cannula.

11. The biopsy marker delivery device of claim 10, further comprising a ramp operatively associated with the side marker exit port.

12. The biopsy marker delivery device of claim 10, further comprising a component disposed around at least a portion of the cannula and slidable with respect to the cannula.

13. A biopsy marker delivery device comprising:
(a) a cannula comprising:
(i) a proximal end,
(ii) a distal end, wherein the distal end comprises a distal tip closing the distal end of the cannula,
(iii) an internal lumen extending from the proximal end to the distal end,
(iv) a side marker exit port disposed proximally of the distal end of the cannula, wherein the side marker exit port is in fluid communication with the internal lumen, wherein the side marker exit portion comprises a first portion and a second portion adjacent to the first portion, wherein the second portion defines a relatively narrow opening extending proximally from the first portion, and
(v) a ramp operatively associated with the side marker exit port; and
(b) at least one marker disposed within the internal lumen of the cannula.
